(19) United States
(12) Patent Application Publication
(10) Pub. No.: US 2019/0224415 A1
(43) Pub. Date: Jul. 25, 2019

(54) Automatic Injection Device With Optimised Layout

(71) Applicant: Nemera La Verpillière, La Verpilliere (FR)

(72) Inventors: Pascal Dugand, Estrablin (FR); Kevin Stamp, Sheffield (GB)

(21) Appl. No.: 16/329,487

(22) PCT Filed: Jul. 25, 2017
(86) PCT No.: PCT/FR2017/052047

§ 371 Date: Feb. 28, 2019

(30) Foreign Application Priority Data
Aug. 31, 2016 (FR) 1658093

(51) Int. Cl. A61M 5/24; A61M 5/20

(52) U.S. Cl. CPC A61M 5/2429; A61M 5/31585; A61M 2005/2073; A61M 5/2033

(57) ABSTRACT

This device is intended to receive an injection syringe. The injection syringe has: a syringe body, a piston mounted slidably in the syringe body, and an injection needle fixed to a distal end of the syringe body. The automatic injection device includes a piston control member, which is displaceable in translation with respect to the syringe body, parallel to an axis X, in order to control the displacement of the piston in the syringe body, a piston rod intended to push a proximal end of the piston of the injection syringe, releasable coupling means for coupling the piston control member and the piston rod, an injection spring which bears on the piston control member and elastically stresses the piston control member towards a distal end of the injection needle. The releasable coupling means are positioned axially between the two axial ends of the injection spring.

### FIELD OF THE INVENTION

This invention relates to the field of automatic injection devices for liquids, especially pharmaceutical.

### BACKGROUND OF THE INVENTION

An automatic injection device is used in particular in the medical field, for automatic administration of a liquid medication requiring an injection. Such a device allows in particular a person, for example suffering from rheumatoid arthritis, multiple sclerosis, diabetes or undergoing an anaphylactic shock in case of allergy, to inject themselves a dose of medication independently.

An automatic injection device intended to receive an injection syringe is already disclosed in the prior art, in particular in U.S. Pat. No. 8,734,402. This injection syringe generally comprises a syringe body, a piston mounted slidably, parallel to an axis X, in the syringe body, and an injection needle fixed to a distal end of the syringe body. This automatic injection device comprises a piston control member, and a piston rod, which are equipped with releasable coupling means. This automatic injection device is equipped with an injection spring intended to stress the control member and displace it, parallel to the axis X, towards the syringe so the piston control member pushes the piston rod and the piston.

In this automatic injection device, the means for coupling the piston control member and the piston rod are positioned axially between the injection syringe and the injection spring. The axial dimension of the automatic injection device is therefore due, at least partly, to the sum of the axial dimensions of the injection syringe, the coupling means and the injection spring.

For reasons of comfort and to minimize the size, it is desirable to limit the length of such an injection device.

### SUMMARY OF THE INVENTION

The invention aims to provide an automatic injection device of reduced length.

To this end, the invention aims to provide an automatic injection device intended to receive an injection syringe, this injection syringe comprising a syringe body, a piston mounted slidably in the syringe body and an injection needle fixed to a distal end of the syringe body, the automatic injection device comprising:

a piston control member, which is displaceable in translation with respect to the syringe body, parallel to an axis X, in order to control the displacement of the piston in the syringe body,

a piston rod intended to push a proximal end of the piston of the injection syringe,

releasable coupling means for coupling the piston control member and the piston rod,

an injection spring which bears on the piston control member and elastically stresses the piston control member towards a distal end of the injection needle,

characterised in that the releasable coupling means are permanently positioned axially between two axial ends of the injection spring.

Thus, the axial dimension of the various members of the injection device between them is optimised and the injection device has a reduced length. The axial dimension of the means for coupling the piston control member and the piston rod has in fact no effect on the axial dimension of the automatic injection device.

According to other optional characteristics of the automatic injection device:

the automatic injection device comprises a first and a second telescopic part whose relative movement controls the actuation of the automatic injection device, the piston control member being mounted slidably in the second telescopic part;

the automatic injection device comprises means for immobilising the piston control member in the second telescopic part comprising at least first and second complementary immobilising stops, the first immobilising stop being retractable under the effect of the relative movement between the first and second telescopic parts;

the first immobilising stop is carried by the second telescopic part.

the first telescopic part is equipped with at least one element for retracting the first immobilising stop actuated under the effect of the relative movement between the first and second telescopic parts;

the first immobilising stop is formed on a radially deformable element carried by the second telescopic part;

the element for retracting the first immobilising stop interacts with a ramp formed on the radially deformable element of the second telescopic part;

the first telescopic part is equipped with at least one element for radial locking of the radially deformable element actuated under the effect of the relative movement between the first and second telescopic parts;

the first immobilising stop is positioned axially between the two axial ends of the injection spring;

the piston rod has a generally tubular shape, a guide element being housed inside the piston rod, the guide element interacting with an inner surface for blocking the piston rod to prevent the piston rod from rotating about its axis X;

the guide element comprises an axial rib interacting with the inner surface for blocking the piston rod to prevent the piston rod from rotating about its axis X.

As those skilled in the art will appreciate, the present invention is not limited to the embodiments, features, and arrangement of parts described above. Other objects of the present invention and its particular features and advantages will become more apparent from consideration of the following drawings and detailed description of the invention.

### BRIEF DESCRIPTION OF THE DRAWINGS

The invention will be better understood on reading the following description, given solely by way of example and with reference to the accompanying drawings in which:

FIG. 1 is a perspective view of an automatic injection device according to one embodiment of the invention, in an initial configuration, before the operation of this automatic injection device;

FIG. 2 is a view similar to FIG. 1, the automatic injection device being shown without an outer casing;

FIG. 3 is a view similar to FIG. 2, in which a control member is shown partially;

FIG. 4 is a view similar to FIG. 3 of a detail of the automatic injection device in which an injection spring is not shown;

FIG. 5 is a perspective view of a member for releasing the automatic injection device shown on FIG. 1;

FIG. 6 is a perspective view of a piston rod of the automatic injection device shown on FIG. 1;

FIG. 7 is a perspective view of a piston control member of the automatic injection device shown on FIG. 1;

FIG. 8 is a sectional view of the injection device along the plane VIII-VIII of FIG. 1;

FIG. 9 is a perspective view with a cross-section of a detail of the outer casing and of the control member of the injection device shown on FIG. 1 assembled together;

FIGS. 10 and 11 are two sectional views of the automatic injection device along planes X-X and XI-XI of FIG. 1;

FIG. 12 is a sectional view similar to FIG. 10 of the automatic injection device shown on FIG. 1 in a configuration depending on the actuation of this automatic injection device.

### DETAILED DESCRIPTION OF THE INVENTION

The following detailed description illustrates the technology by way of example, not by way of limitation of the principles of the invention. This description will enable one skilled in the art to make and use the technology, and describes several embodiments, adaptations, variations, alternatives and uses of the invention, including what is presently believed to be the best mode of carrying out the invention. One skilled in the art will recognize alternative embodiments, features, and arrangements, and the present technology is not limited to those described hereafter.

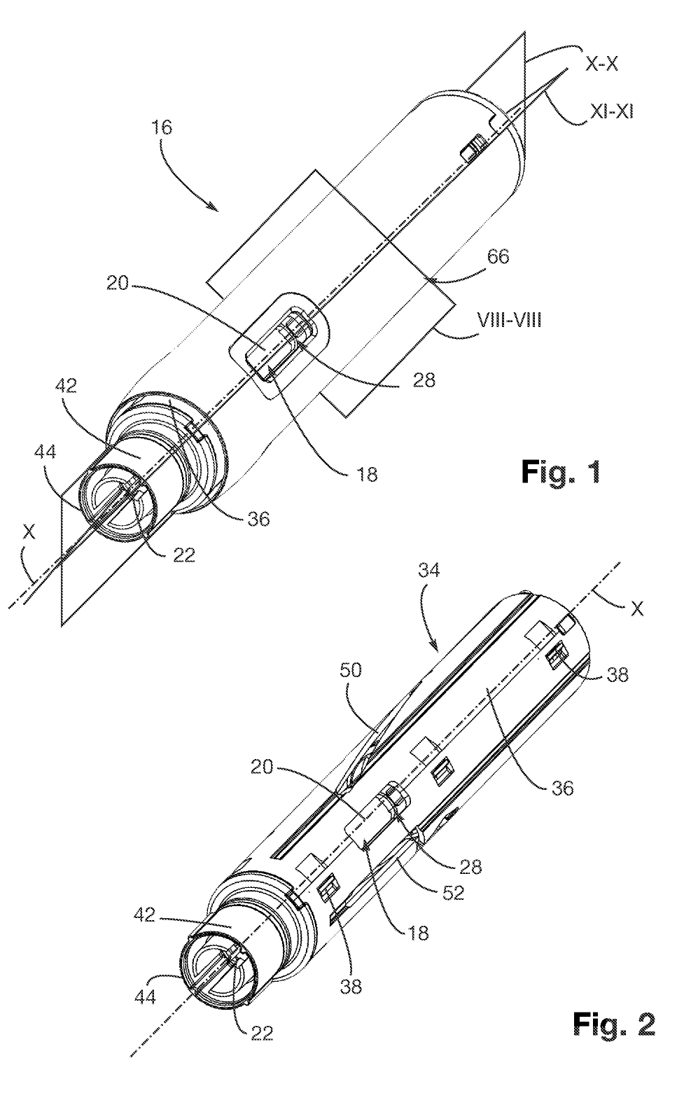

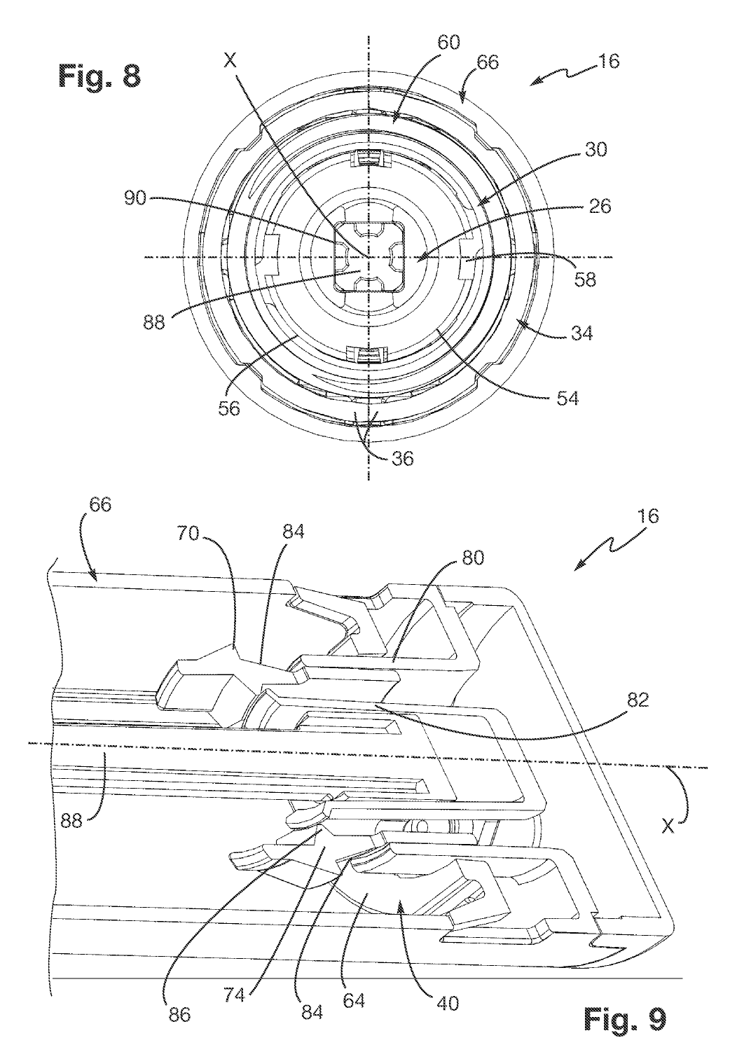

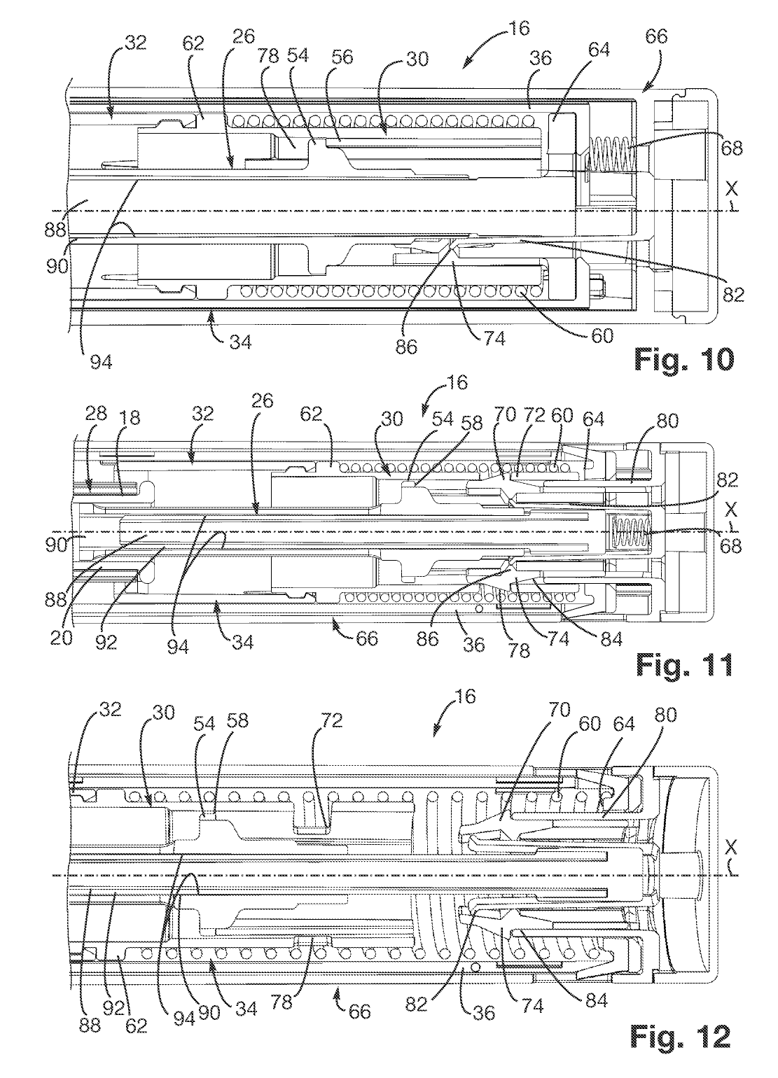

FIGS. 1 to 12 show an automatic injection device 16 according to the invention. This automatic injection device 16 has a general shape of revolution about the axis X. This automatic injection device 16 is intended to be manually grasped at one end, which will be referred to subsequently as the proximal end. The opposite end, which will be referred to as the distal end, is intended to be applied against a patients skin, on an injection area.

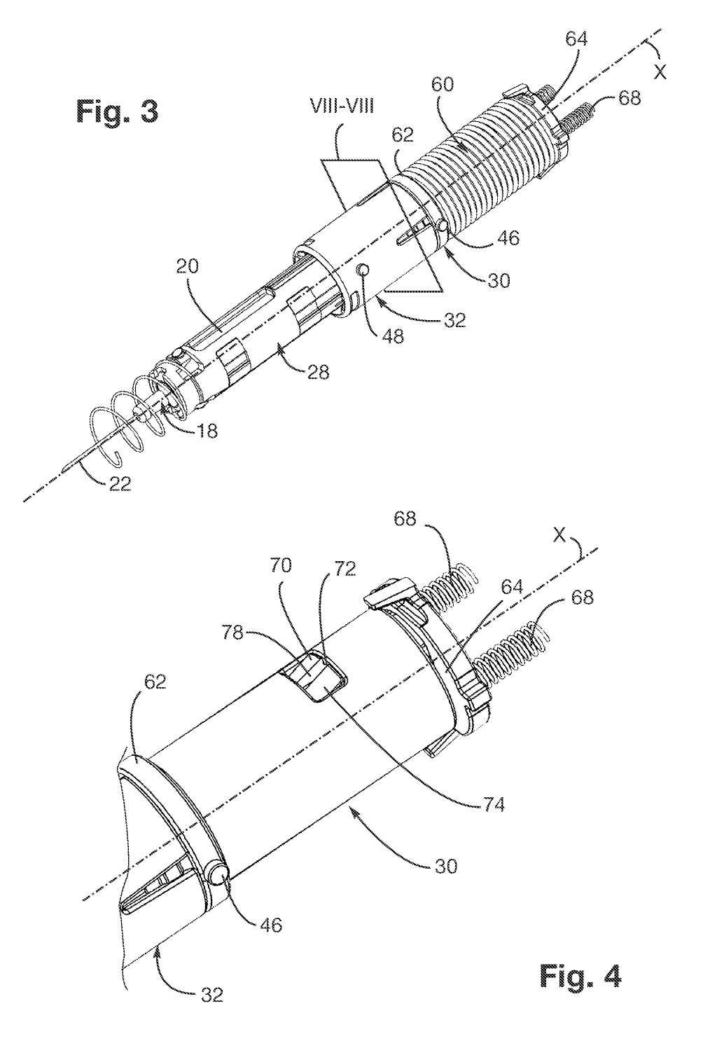

The automatic injection device 16 is intended to be equipped with an injection syringe 18, shown in particular on FIG. 3. In this embodiment of the invention, the injection syringe 18 is a traditional injection syringe 18 made of glass comprising a syringe body 20 of generally tubular shape of axis X containing a pharmaceutical liquid, which may be relatively viscous, to be injected in the patients body.

The distal end of the syringe body 20 is equipped with an injection needle 22. A piston (traditional and not shown) is mounted slidably in the syringe body 20 so that when the piston moves towards the distal end of the syringe body 20, the pharmaceutical liquid contained in the syringe body 20 is ejected through the injection needle 22.

The injection syringe 18 is equipped with a traditional cap (not shown on the figures) for protecting the injection needle 22, covering the injection needle 22. This cap must be removed before using the automatic injection device 16.

A piston rod 26, shown on FIG. 6, is in contact with a proximal end of the piston. This piston rod 26 is intended to push the piston towards the distal end of the syringe body 20 when injecting the pharmaceutical product into the patients body.

The automatic injection device 16 comprises a syringe support 28, shown in particular on FIG. 3, for carrying the injection syringe 18. The syringe support 28 has a generally tubular shape of axis X comprising an opening at the distal end and at the proximal end. When operating the automatic injection device 16, the injection syringe 18 is fixed in the syringe support 28.

A piston control member 30 and a needle control member 32, shown in particular on FIGS. 3 and 4, are housed in a proximal part of the syringe support 28 and are displaceable in translation with respect to the syringe body 20, parallel to the axis X. The syringe support 28 and the control members 30, 32 are mounted slidably in a positioning control member 34 of generally tubular shape of axis X, shown in particular on FIG. 2. The control members 30, 32 have a generally tubular shape of axis X.

The positioning control member 34 comprises:

two half-shells 36 attached together by traditional clipping means 38,

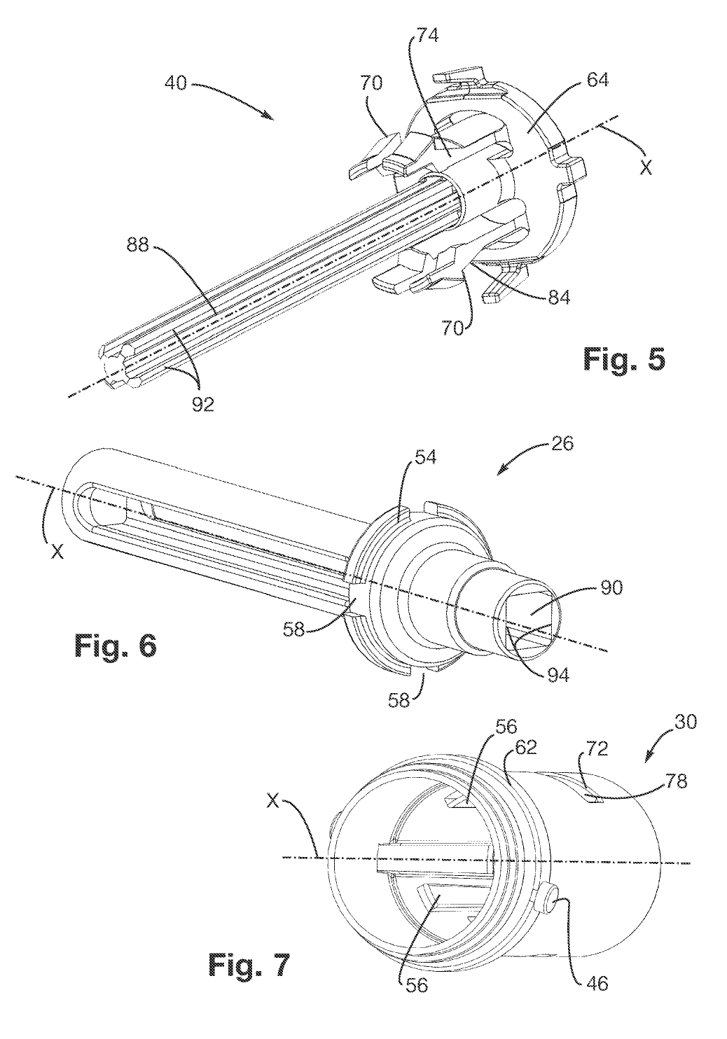

a retaining member 40, positioned in the proximal part of the positioning control member 34, shown on FIG. 5, whose purpose will be specified below,

an end sleeve 42 attached to the distal end of the two half-shells 36.

The end sleeve 42 is equipped with a distal surface 44 which is intended to be brought into contact with the patient's skin.

The piston control member 30 is equipped with a first pair of diametrically opposed cams 46 and the needle control member 32 is equipped with a second pair of diametrically opposed cams 48. These first and second pairs of cams 46, 48 interact respectively with first and second pairs of camways 50, 52 formed in the half-shells 36 of the positioning control member 34 (see FIG. 2). By interacting with the first and second pairs of camways 50, 52, the control members 30, 32 rotate about the axis X between several configurations which will be described below.

The piston control member 30 and the piston rod 26 are equipped with releasable coupling means 54, 56 (see FIGS. 6 to 8). These releasable coupling means comprise a shoulder 54 formed in the proximal part of the piston rod 26 and four axial ribs 56 formed inside the piston control member 30. Four notches 58 are formed in the shoulder 54 of the piston rod 26. When operating the automatic injection device 16, the piston control member 30 and the piston rod 26 can be positioned relative to one another in two configurations:

an engaged configuration, in which the ends of the axial ribs 56 of the piston control member 30 interact with the shoulder 54 of the piston control member 30, and

a release configuration, in which the notches 58 formed in the shoulder 54 of the piston rod 26 are opposite the ribs 56 of the piston control member 30, the piston rod 26 then being able to move towards the proximal end of the piston control member 30 by retracting axially in the piston control member 30.

The piston rod 26 and the piston control member 30 move from their engaged configuration to their release configuration by rotating relative to one another about the axis X, during the axial displacement of the piston control member 30 in the positioning control member 34. This rotation is caused by the interaction of the first pair of cams 46 with the first pair of camways 50.

Similarly, the needle control member 32 and the syringe support 28 are equipped with means adapted to move from an engagement configuration to a release configuration when the needle control member 32 and the syringe support 28 rotate relative to one another about the axis X.

An injection spring 60, shown in particular on FIG. 3, is housed in the proximal part of the automatic injection device 16. The injection spring 60 is a compression spring compressed between a shoulder 62 formed on the distal end of the piston control member 30 and a seat 64 of the injection spring 60 formed in the proximal end of the member 40 for retaining the positioning control device 34.

The injection spring 60 has a generally helical shape whose inner diameter is sufficient for the releasable coupling means 54, 56 of the piston control member 30 and of the piston rod 26 to be housed inside the injection spring 60. The releasable coupling means 54, 56 are axially closer to the proximal end of the positioning control member 34 than the distal end of the injection spring 60. Thus, the releasable coupling means 54, 56 are positioned axially between two axial ends of the injection spring 60.

As can be seen in particular on FIGS. 1 and 10 to 12, actuation of the automatic injection device 16 is controlled by the relative movement of a first and a second telescopic part.

The first telescopic part comprises an outer casing 66. The outer casing 66 has a generally tubular shape of axis X and is mounted slidably around the second telescopic part. The second telescopic part comprises the positioning control member 34. Return springs 68 are housed in the distal end of the outer casing 66. The return springs 68 are compression springs which return the positioning control member 34 axially toward the distal end of the outer casing 66.

During actuation, the positioning control member 34 moves axially, towards the proximal end of the outer casing 66, relative to the outer casing 66, from an initial position, in which the automatic injection device 16 is not actuated, to an actuation position, in which the automatic injection device 16 is actuated.

As can be seen in particular on FIG. 4, the retaining member 40 and the piston control member 30 comprise means 70, 72 for immobilising the piston control member 30 in the positioning control member 34. These immobilising means comprise a first pair of retractable immobilising stops 70 complementary with a second pair of immobilising stops 72.

The retaining member 40, which is part of the positioning control member 34, comprises a pair of radially deformable elements 74 on which the first pair of immobilising stops 70 is formed. These deformable elements 74 are two diametrically opposed flexible tabs 74.

The second retractable stops 72 are formed by edges of two diametrically opposed windows 78 formed in the piston control member 30.

The flexible tabs 74 can be deformed between a rest position in which they are radially away from the axis X, and a retracted position in which they are radially close to the axis X. When the flexible tabs 74 are in their rest position, the first and second pairs of immobilising stops 70, 72 interact with one another and the piston control member 30 and the positioning control member 34 are immobilised relative to one another. When the flexible tabs 74 are in their retracted position, the first and second pairs of immobilising stops 70, 72 no longer interact with one another, the piston control member 30 and the positioning control member 34 then no longer being immobilised axially relative to one another.

The outer casing 66 is equipped, for each flexible tab 74, with a retraction element 80 (see in particular FIG. 9) and a radial locking element 82.

The retraction element 80 is a tab whose distal end interacts with a ramp 84 formed on the flexible tab 74, when the positioning control member 34 moves axially towards the proximal end of the outer casing 66. By interacting with the ramp 84, the retraction element 80 deforms the flexible tab 74 from its rest position to its retraction position.

The radial locking element 82 is a tab whose distal end is intended to interact with an inner radial lug 86 formed on the flexible tab 74 when the positioning control member 34 is in its initial configuration. The radial locking element 82 is positioned so that it prevents any radial displacement of the flexible tab 74 when the positioning control member 34 is in its initial position. This immobilisation is intended to prevent any inadvertent operation of the automatic injection device 16, in particular in case of impact which could briefly deform the flexible tabs 74 to their release position.

The fact that the flexible tabs 74 and the retraction elements 80 are carried by the first and second telescopic parts 66, 34 controlling actuation of the automatic injection device 16 prevents the sudden release of the flexible tabs 74 when actuating the automatic injection device 16, which is the case in particular if the flexible tabs 74 are formed on the piston control member 30. In fact, after actuating the automatic injection device 16, the piston control member 30 moves quickly toward the distal end of the automatic injection device 16, which is not the case of the second telescopic part 34 which remains in its actuation position until the last operating phase of the automatic injection device 16.

The seat 64 of the injection spring 60 is closer to the distal end of the automatic injection device 16 than the first immobilising stop 70, so that the first immobilising stop 70 is positioned axially between the two axial ends of the injection spring 60.

The retaining member 40 is equipped with an element 88 for guiding the piston rod 26. The guide element 88 is a guide rod 88 extending axially. The piston rod 26 has a generally tubular shape. The guide element 88 is mounted slidably in an axial hole 90 formed in the piston rod 26 of generally square cross-section. The guide rod 88 is equipped with four axial ribs 92 distributed circumferentially. These axial ribs 92 interact with inner blocking surfaces 94 forming contours of the axial hole 90 of the piston rod 26 so as to prevent any rotation of the piston rod 26 relative to the positioning control member 34. The guide rod may also have a square, triangular, rectangular cross-section or any other suitable shape, or have one or more reliefs other than axial ribs and preventing its rotation relative to the control member.

The various operating steps of the automatic injection device 16 according to the invention will be described below.

To actuate the automatic injection device 16, the user grasps the outer casing 66 and applies the distal surface 44 of the end sleeve 42 against the patients skin then presses the automatic injection device 16 briefly against the patients skin so as to slide the positioning control member 34 in the outer casing 66.

When the positioning control member 34 slides in the outer casing 66j, the ramps 84 of the flexible tabs 74 interact with the retraction element 80 of the outer casing 66 and move to their retracted configuration (see FIG. 12).

When the positioning control member 34 reaches its position for actuating the automatic injection device 16, the first and second complementary immobilising stops 70, 72 no longer interact and the piston control member 30 is free to move axially relative to the positioning control member 34.

Thus, under the action of the injection spring 60, the piston control member 30 moves towards the distal end of the positioning control member 34. The needle control member 32 is driven by the piston control member 34 and pushes the syringe support 28 and the injection syringe 18 so that the injection needle 22 is inserted into the patients skin.

When the injection needle 22 has been inserted, the syringe support 28 and the needle control member 32 move from their engaged configuration to their release configuration.

The needle control member 32 and the piston control member 30 can then continue their axial movement towards the distal end of the positioning control member 34 without driving the syringe support 28. The piston control member 30, which is still engaged with the piston rod 26, pushes the piston rod 26 and the piston towards the distal end of the automatic injection device 16. Thus, the pharmaceutical liquid contained in the injection syringe 18 is injected.

When the injection is finished, the piston control member 30 and the piston rod 26 move from their engaged configuration to their release configuration. Thus, the piston rod 26, the injection syringe 18 and the syringe support 28 are free to move axially towards the proximal end of the positioning control member 34. Return means known by those skilled in the art push the injection syringe 18 towards the proximal end of the positioning control member 34 so that the injection needle 22 retracts.

Thus, from the initial position before actuation of the automatic injection device 16 to the final position in which the needle 22 retracts, the releasable coupling means 54, 56 are permanently positioned axially between two axial ends of the injection spring 60. Consequently, the axial dimension of the various members of the automatic injection device 16 between them is optimised and the automatic injection device 16 has a reduced length. The axial dimension of the means 54, 56 for coupling the piston control member 30 and the piston rod 26 has in fact no effect on the axial dimension of the automatic injection device 16.

The invention is not limited to the embodiments described, and other embodiments will be clearly apparent to those skilled in the art. It is in particular possible to modify the configuration of the control member camways to reduce their axial dimensions. Other modifications and variations of the invention's features and arrangements thereof will be ascertainable to those of ordinary skill in the art.

1. Automatic injection device intended to receive an injection syringe, this injection syringe comprising a syringe body, a piston mounted slidably in the syringe body and an injection needle fixed to a distal end of the syringe body, the automatic injection device comprising:
a piston control member, which is displaceable in translation with respect to the syringe body, parallel to an axis X, in order to control the displacement of the piston in the syringe body,
a piston rod intended to push a proximal end of the piston of the injection syringe,
releasable coupling means for coupling the piston control member and the piston rod,
an injection spring which bears on the piston control member and elastically stresses the piston control member towards a distal end of the injection needle,
characterised in that the releasable coupling means are permanently positioned axially between two axial ends of the injection spring.

2. Automatic injection device according to claim 1, comprising a first telescopic part and a second telescopic part whose relative movement controls the actuation of the automatic injection device, the piston control member being mounted slidably in the second telescopic part.

3. Automatic injection device according to claim 2, comprising means for immobilising the piston control member in the second telescopic part comprising at least a first immobilizing stop and a second complementary immobilising stop, the first immobilising stop being retractable under the effect of the relative movement between the first and second telescopic parts.

4. Automatic injection device according to claim 3, wherein the first immobilising stop is carried by the second telescopic part.

5. Automatic injection device according to claim 4, wherein the first telescopic part is equipped with at least one element for retracting the first immobilising stop actuated under the effect of the relative movement between the first and second telescopic parts.

6. Automatic injection device according to claim 4, wherein the first immobilising stop is formed on a radially deformable element carried by the second telescopic part.

7. Automatic injection device according to claim, wherein the element for retracting the first immobilising stop interacts with a ramp formed on the radially deformable element of the second telescopic part.

8. Automatic injection device according to claim 6, wherein the first telescopic part is equipped with at least one element for radial locking of the radially deformable element actuated under the effect of the relative movement between the first and second telescopic parts.

9. Automatic injection device according to claim 3, wherein the first immobilising stop is positioned axially between the two axial ends of the injection spring.

10. Automatic injection device according to claim 1, wherein the piston rod has a generally tubular shape and a guide element is housed inside the piston rod, the guide element interacting with an inner surface for blocking the piston rod to prevent the piston rod from rotating about its axis X.

11. Automatic injection device according to claim 10, wherein the guide element comprises an axial rib interacting with the inner surface for blocking the piston rod to prevent the piston rod from rotating about its axis X.
